(19) United States
(12) Patent Application Publication
(10) Pub. No.: US 2022/0244261 A1
(43) Pub. Date: Aug. 4, 2022

(54) METHOD FOR DIAGNOSING COLORECTAL CANCER

(71) Applicant: AB Mavatar, Goteborg (SE)

(72) Inventor: Danuta Gawel, Atvidaberg (SE)

(73) Assignee: AB Mavatar, Goteborg (SE)

(21) Appl. No.: 17/614,887

(22) PCT Filed: May 29, 2020
(86) PCT No.: PCT/EP2020/065062

§ 371 Date: Nov. 29, 2021

(30) Foreign Application Priority Data
May 30, 2019 (GB) 1907663.7

(51) Int. Cl. G01N 33/574

(52) U.S. Cl. CPC G01N 33/57419; G01N 2470/06

(57) ABSTRACT

The present invention provides a non-invasive method of diagnosing colorectal cancer in a subject. The method comprises determining the blood concentrations of the proteins TRIM28, PLOD1 and CEACAM5 (and optionally P4HA1) in a subject. An analysis of the concentrations is performed to determine whether the subject has colorectal cancer.

The present invention relates to methods for diagnosing colorectal cancer in a subject, extending to methods for diagnosing and treating colorectal cancer in a subject. The invention also provides a kit suitable for use in the diagnostic assay of the invention, and a computer programme for automated performance of the diagnostic assay.

Colorectal cancer (CRC) is the third most common form of cancer. Globally, it affects more than 1.2 million individuals each year, and causes some 700,000 deaths. Survival rates have improved over the last 30 years due to better diagnostics, surgical and oncological treatment. Survival is, however, still most related to the tumour stage at diagnosis. At stage I, the 5-year survival rate is 87-92%; at stage II it's 49-87%, at stage III 53-89%, and at Stage IV 11-12%. It is therefore essential to identify colorectal tumours as early as possible, to provide the best prospect for survival. This has led to recommendations for national screening programmes in many countries. These programmes are mainly based on detecting occult blood in faeces, followed by colonoscopy (and optional biopsy) to confirm the diagnosis. This has increased early detection of CRC and reduced mortality. A problem, however, is that screening programmes yield a large number of false positives, resulting in many healthy individuals being subjected to unnecessary colonoscopy. This investigation is costly and perceived as unpleasant or painful by many subjects. Furthermore, colonoscopy is associated with a small risk of bowel perforation. Thus, there is a need for improved and more accurate biomarkers which allow early diagnosis of CRC while reducing the number of false positives in initial screening, reducing the number of unnecessary colonoscopies performed. Several such biomarkers have been proposed, including combinations of multiple proteins (see e.g. Banger et al., BMC Cancer 2012, 12:393), microRNAs (Nana-Sinkam et al., Ann N Y Acad Sci 2010, 1210: 25-33), DNA methylation (e.g. Warren et al., BMC Medicine 2011, 9:133) or tumour DNA in blood or stools (e.g. Petit et al., J Surg Res 2019, 236: 184-197).

However, in general these proposed biomarker-based diagnostic tests are not used in clinical settings. Newer tests have not been adopted for a variety of reasons, such as excessive complexity of measurement, or a failure to satisfactorily demonstrate their accuracy. Nonetheless, one important observation from the studies performed to date is that tests based on combinations of biomarkers offer superior diagnostic accuracy relative to tests performed using individual ones (see e.g. Nystrom et al., Tumour Biol 2015, 36:9839-47). This suggests that a standard diagnostic assay based on a limited number of highly accurate biomarkers that can be measured in routine clinical settings is required. However, the scale of the problem in developing such an assay is shown by a recent survey of the medical literature, which identified 383 proteins, 94 mRNAs, 35 DNAs and 185 other forms of potential biomarkers for CRC (Zhang et al., Database 2018, Bay046).

The present inventors have developed such a standard diagnostic assay suitable for use in routine clinical settings. The assay is based on combinations of 3 or 4 plasma protein biomarkers, some of which are known CRC biomarkers and others of which have not previously been shown to be associated with CRC. According to the inventors' new assay, CRC can be diagnosed based simply on the plasma concentrations of the protein biomarkers. The test is thus non-invasive, can be performed rapidly using routine clinical methods and instruments and, as shown in the Examples, is highly accurate. The test may also be automated. Use of the test in standard screening programmes has the potential to significantly improve the accuracy of the screening programmes, reducing false positives and thus reducing the number of unnecessary colonoscopies performed, reducing burdens on healthcare systems and allowing healthy individuals to avoid the risks associated with colonoscopy.

Thus, in a first aspect the invention provides a method of diagnosing colorectal cancer in a subject, comprising determining the concentrations of the proteins TRIM28, PLOD1 and CEACAM5 in a blood-derived sample from the subject, and based on the summed concentrations and/or relative concentrations of said proteins determining whether the subject is suffering from colorectal cancer.

In a second aspect, the invention provides a method of diagnosing colorectal cancer in a subject, comprising determining the concentrations of the proteins TRIM28, PLOD1, CEACAM5 and P4HA1 in a blood-derived sample from the subject, and based on the summed concentrations and/or relative concentrations of said proteins determining whether the subject is suffering from colorectal cancer.

In a third aspect, the invention provides a method of diagnosing and treating colorectal cancer in a subject, comprising performing a method of diagnosing colorectal cancer according to the first or second aspect of the invention, and when a subject is diagnosed with colorectal cancer, administering treatment for colorectal cancer to the subject.

In a fourth aspect the invention provides a kit comprising a set of reagents for determining the presence or concentration of TRIM28, PLOD1, CEACAM5 and, optionally, P4HA1 in a sample.

Such reagents may comprise binding agents, such as antibodies, capable of binding specifically to TRIM28, PLOD1, CEACAM5 or P4HA1 and distinguishing it from another protein. A reagent for determining the presence or concentration of a particular protein is able to report on its presence or absence in a sample, or more particularly may be able to quantify the amount of the protein that is present in the sample. Such a reagent may be defined as specific for the protein.

In a fifth aspect the invention provides a computer programme product comprising instructions that, when executed, will cause a processor to perform a method according to the first or second aspect.

In a sixth aspect the invention provides the use of a kit of the fourth aspect in the diagnosis of colorectal cancer, wherein said diagnosis is performed using a method according to the first or second aspect.

Thus the present application provides methods of diagnosing colorectal cancer in a subject. Colorectal cancer is also referred to as bowel cancer, and in some instances colon cancer and rectal cancer are referred to as separate cancers. As used herein, in accordance with standard practice in the art, colorectal cancer encompasses any cancer originating from the tissue of the colon or the rectum. Thus, colorectal cancer includes primary colorectal cancer, i.e. colorectal cancer located at its original site of development within the colon or rectum, and secondary colorectal cancer, i.e. metastases of colorectal cancer located elsewhere in the body. In accordance with standard practice, secondary tumours present in the colon or rectum but originating from elsewhere in the body are not considered colorectal cancers.

The methods of the invention may be used to diagnose any type of colorectal cancer, including adenocarcinoma, squamous cell carcinoma and adenosquamous carcinoma, sarcoma, carcinoid tumours and lymphoma. Similarly, the methods of the invention may be used to diagnose colorectal cancer of any stage, including stage I, stage II, stage III and stage IV colorectal cancer. The Dukes' staging system may also be used to describe colorectal cancer progression, and equivalently the methods of the invention may be used to diagnose colorectal cancer at Dukes' stage A, Dukes' stage B, Dukes' stage C or Dukes' stage D.

The subject for whom the methods are performed is a human subject. The subject may be male or female, of any age group or ethnicity. The method may be used for colorectal cancer diagnosis in a subject experiencing symptoms of colorectal cancer, a subject considered to be at risk of colorectal cancer, or an apparently healthy subject. For instance, the method may be used in a universal screening programme to screen for colorectal cancer across a population. The method may also be used to screen for recurrence of colorectal cancer in a subject who has previously been successfully treated for the disease.

The term “diagnosing” as used herein can be understood as determining whether a subject does or does not have colorectal cancer. Thus, the method can be used to determine that a subject has (or probably has) colorectal cancer, that a subject being screened for colorectal cancer does not have (or probably does not have) colorectal cancer, or to rule out colorectal cancer in an individual suspected of having colorectal cancer. Notably, while the invention provides methods for diagnosing colorectal cancer, the use of these methods does not preclude the performance of additional diagnostic procedures to confirm a diagnosis made based upon a method of the invention. For instance, it is envisaged that individuals indicated by the methods of the invention as having colorectal cancer will undergo a confirmatory colonoscopy to confirm the cancer diagnosis, and possible a biopsy. It is also possible that the diagnostic methods of the invention could be used in combination with a faecal occult blood test in population screening programmes.

The methods of the invention comprise determining the concentrations of the proteins TRIM28, PLOD1 and CEACAM5 in a blood-derived sample from the subject. TRIM28 is encoded by the TRIM28 gene and is alternatively known as TIF1ß or KAP1. TRIM28 has the UniProt accession number Q13263 and the amino acid sequence set forth in SEQ ID NO: 1. PLOD1 is encoded by the PLOD1 gene, and is alternatively known as LH1. PLOD 1 has the UniProt accession number Q02809 and the amino acid sequence set forth in SEQ ID NO: 2. CEACAM5 is encoded by the CEACAM5 gene and is alternatively known as CEA, meconium antigen 100 and CD66e. CEACAM5 has the UniProt accession number P06731 and the amino acid sequence set forth in SEQ ID NO: 3.

The methods of the invention may further comprise determining the concentrations of the protein P4HA1 in the blood-derived sample from the subject. P4HA1 is encoded by the P4HA1 gene and has the UniProt accession number P13674. The amino acid sequence of P4HA1 is set forth in SEQ ID NO: 4. The proteins used in the methods of the invention are referred to herein as “proteins of interest”. They may alternatively be referred to as biomarkers.

As noted above, the subject is a human and thus all references herein to TRIM28, PLOD1, CEACAM5 and P4HA1 are to human TRIM28, PLOD1, CEACAM5 and P4HA1, respectively.

CEACAM5 may promote tumour development by acting as a cell adhesion molecule, and by regulating differentiation, apoptosis, and cell polarity; TRIM28 is a transcriptional corepressor, with complex and context-dependent effects. For example, in a mouse model of liver cancer TRIM28 opposes tumourigenesis (Herquel et al., Proc Natl Acad Sci USA 2011, 108:8212-8217); PLOD1 has an important role in extracellular matrix formation (Wang et al., Genet Test Mol Bioma 2018, 22:366-373); P4HA1 regulates collagen formation, and has been implicated in melanoma (Atkinson et al., J Invest Dermatol 2019, 139(5):1118-1126). CEACAM5 and TRIM28 have previously been identified as potential blood biomarkers for CRC (Shiromizu et al., Sci Rep 2017, 7:12782).

The skilled person will appreciate that the amino acid sequences of one or more of TRIM28, PLOD1, CEACAM5 and P4HA1 may differ slightly from those presented in SEQ ID NOs: 1-4 due to natural variation, such as single nucleotide polymorphisms (SNPs) in the encoding genes, mutations or alternative splicing of mRNA. In case of doubt, TRIM28 is defined herein as encompassing any protein which has an amino acid sequence with at least 90% sequence identity to the amino acid sequence set forth in SEQ ID NO: 1 and which is recognised by an antibody which specifically binds a protein of SEQ ID NO: 1. TRIM28 may alternatively be defined as encompassing any protein which has an amino acid sequence with at least 95%, 98% or 99% sequence identity to the amino acid sequence set forth in SEQ ID NO: 1 and which is recognised by an antibody which specifically binds a protein of SEQ ID NO: 1. In a further alternative, TRIM28 is defined as the protein of SEQ ID NO: 1.

Similarly, PLOD1 is defined herein as encompassing any protein which has an amino acid sequence with at least 90% sequence identity to the amino acid sequence set forth in SEQ ID NO: 2 and which is recognised by an antibody which specifically binds a protein of SEQ ID NO: 2. PLOD1 may alternatively be defined as encompassing any protein which has an amino acid sequence with at least 95%, 98% or 99% sequence identity to the amino acid sequence set forth in SEQ ID NO: 2 and which is recognised by an antibody which specifically binds a protein of SEQ ID NO: 2. In a further alternative, PLOD1 is defined as the protein of SEQ ID NO: 2.

CEACAM5 is defined herein as encompassing any protein which has an amino acid sequence with at least 90% sequence identity to the amino acid sequence set forth in SEQ ID NO: 3 and which is recognised by an antibody which specifically binds a protein of SEQ ID NO: 3. CEACAM5 may alternatively be defined as encompassing any protein which has an amino acid sequence with at least 95%, 98% or 99% sequence identity to the amino acid sequence set forth in SEQ ID NO: 3 and which is recognised by an antibody which specifically binds a protein of SEQ ID NO: 3. In a further alternative, CEACAM5 is defined as the protein of SEQ ID NO: 3.

P4HA1 is defined herein as encompassing any protein which has an amino acid sequence with at least 90% sequence identity to the amino acid sequence set forth in SEQ ID NO: 4 and which is recognised by an antibody which specifically binds a protein of SEQ ID NO: 4. P4HA1 may alternatively be defined as encompassing any protein which has an amino acid sequence with at least 95%, 98% or 99% sequence identity to the amino acid sequence set forth in SEQ ID NO: 4 and which is recognised by an antibody which specifically binds a protein of SEQ ID NO: 4. In a further alternative, P4HA1 is defined as the protein of SEQ ID NO: 4.

Sequence identity may be assessed by any convenient method. However, for determining the degree of sequence identity between sequences, computer programmes that make pairwise or multiple alignments of sequences are useful, for instance EMBOSS Needle or EMBOSS stretcher (both Rice, P. et al., Trends Genet., 16, (6) pp 276-277, 2000) may be used for pairwise sequence alignments while Clustal Omega (Sievers F et al., Mol. Syst. Biol. 7:539, 2011) or MUSCLE (Edgar, R. C., Nucleic Acids Res. 32(5):1792-1797, 2004) may be used for multiple sequence alignments, though any other appropriate programme may be used. Whether the alignment is pairwise or multiple, it must be performed globally (i.e. across the entirety of the reference sequence) rather than locally.

Sequence alignments and % identity calculations may be determined using for instance standard Clustal Omega parameters: matrix Gonnet, gap opening penalty 6, gap extension penalty 1. Alternatively the standard EMBOSS Needle parameters may be used: matrix BLOSUM62, gap opening penalty 10, gap extension penalty 0.5. Any other suitable parameters may alternatively be used.

Herein, an entity (e.g. molecule) which “specifically binds” another is known as a “specific binding agent”. A specific binding agent is an agent (i.e. molecule) which binds specifically to a particular binding partner. More particularly, a specific binding agent is capable of binding to its target in a manner which may be distinguished from binding to a non-target molecule. Thus, binding to a non-target molecule may be negligible or substantially reduced as compared to binding to a target molecule. An antibody is an example of a specific binding agent.

A specific binding agent (e.g. antibody) which specifically binds a protein of SEQ ID NO: 1 (i.e. TRIM28) may be a molecule which binds to TRIM28 with a greater affinity than that with which it binds to other molecules, or at least most other molecules. Thus, for example, if a specific binding agent which binds TRIM28 were contacted with a lysate of human cells, the specific binding agent would bind primarily to TRIM28. In particular, the specific binding agent binds to a sequence or configuration present on TRIM28, preferably a unique sequence or configuration not present on other molecules. When the specific binding agent is an antibody the sequence or configuration is the epitope to which the antibody binds. A specific binding agent which binds TRIM28 does not necessarily bind only to TRIM28: the specific binding agent may cross-react with certain other undefined target molecules, or may display a level of non-specific binding when contacted with a mixture of a large number of molecules (such as a cell lysate or suchlike). However, the skilled person will easily be able to identify whether a specific binding agent shows specificity for TRIM28 using standard techniques in the art, e.g. ELISA, Western-blot, surface plasmon resonance (SPR), etc. Similarly, a specific binding agent which binds a protein of SEQ ID NO: 2 (i.e. PLOD1) may be a molecule (e.g. antibody) which binds to PLOD1 with a greater affinity than that with which it binds to other molecules, or at least most other molecules; a specific binding agent which binds a protein of SEQ ID NO: 3 (i.e. CEACAM5) may be a molecule (e.g. antibody) which binds to CEACAM5 with a greater affinity than that with which it binds to other molecules, or at least most other molecules; and a specific binding agent which binds a protein of SEQ ID NO: 4 (i.e. P4HA1) may be a molecule (e.g. antibody) which binds to P4HA1 with a greater affinity than that with which it binds to other molecules, or at least most other molecules.

As defined herein, a protein is “recognised” by an antibody or other binding molecule if it is specifically bound by that antibody or other binding molecule. A specific binding agent may alternatively be defined as a specific binding partner for a given protein. As mentioned above, whether an antibody specifically binds a particular molecule, e.g. protein, can be determined by standard techniques in the art, e.g. using ELISA, Western-blot or SPR, etc.

The term “antibody” is used herein to refer broadly to any and all types of antibody molecule or antibody fragment. The term thus includes any molecule which is a full-length immunoglobulin molecule, or a fragment or derivative thereof. Accordingly, subsumed under this term is any antibody-type or antibody-derived molecule or fragment, or more generally any molecule which comprises an antigen-binding domain derived or obtained from an antibody (e.g. an immunoglobulin molecule, such as a native antibody), or based on the antigen-binding domain of an antibody. An antibody may alternatively be defined as immunological binding agent, or an immunointeractive agent.

A full-length immunoglobulin molecule comprises two full-length heavy chains and two light chains. Typically, the heavy chains are identical to each other and the light chains are identical to each other. The light chains are shorter (and thus lighter) than the heavy chains. The heavy chains comprise four or five domains: at the N-terminus a variable (VH) domain is located, followed by three or four constant domains (from N-terminus to C-terminus C_{H}1, C_{H}2, C_{H}3 and, where present, C_{H}4, respectively). The light chains comprise two domains: at the N-terminus a variable (V_{L}) domain is located and at the C-terminus a constant (C_{L}) domain is located. In the heavy chain an unstructured hinge region is located between the C_{H}1 and C_{H}2 domains. The two heavy chains of an antibody are joined by disulphide bonds formed between cysteine residues present in the hinge region, and each heavy chain is joined to one light chain by a disulphide bond between cysteine residues present in the C_{H}1 and C_{L} domains, respectively.

In mammals two types of light chain are produced, known as lambda (λ) and kappa (κ). For kappa light chains, the variable and constant domains can be referred to as V_{K} and C_{K} domains, respectively. Whether a light chain is a λ or κ light chain is determined by its constant region: the constant regions of λ and κ light chains differ, but are the same in all light chains of the same type in any given species. The constant regions of the heavy chains are the same in all antibodies of any given isotype in a species, but differ between isotypes (examples of antibody isotypes are classes IgG, IgE, IgM, IgA and IgD; there are also a number of antibody sub-types, e.g. there are four sub-types of IgG antibodies: IgG1, IgG2, IgG3 and IgG4). The specificity of an antibody is determined by the sequence of its variable region.

An antibody may accordingly be of any desired or convenient species, class or sub-type, It may be natural, derivatised or synthetic. Poly- or monoclonal antibodies are included, and any fragment thereof, as described further below. Antibody derivatives, such as single chain antibodies, chimeric antibodies and other synthetically made or altered antibody-like molecules are described further below, and all are included.

By “blood-derived sample from the subject” is meant any sample derived from the blood of the subject. The sample may be whole blood (also referred to herein simply as “blood”) as obtained from the subject, without any additional processing, for example fractionation, or it may be a processed sample, e.g. a fraction of a whole blood sample. Processing may also include other steps. For example, the blood sample, or fraction thereof, may or may not be subjected to, for example, contacting with various agents, such as anticoagulants or any other component which may be present in a blood collection vessel.

Blood may be obtained from the subject using standard clinical methods, e.g. venipuncture (phlebotomy), fingerprick or heelprick. The blood sample is likely to be a venous blood sample, but arterial blood is equally suitable. Any suitable method may be used to obtain a sample of blood from the subject, as known to skilled practitioners in the art such as clinicians, nurses and phlebotomists. In a particular embodiment, the methods of invention further comprise taking a blood sample from the subject. The blood sample may be taken from the subject using the techniques listed above. Notably, the step of taking a blood sample from the subject may be, and indeed is likely to be, performed by a different individual to the individual who performs the diagnostic method upon the blood-derived sample. For instance, as mentioned above, the blood sample may be taken by e.g. a physician, nurse or phlebotomist, while the diagnostic method described below may be performed by e.g. a pathologist or clinical biochemist.

The blood-derived sample may be a sample obtained by processing of a blood sample obtained from the subject. The methods of the invention may further comprise processing a blood sample from the subject to yield a blood-derived sample. As noted above, such processing may include fractionation, and in particularly obtaining a fraction of blood from which blood cells have been removed. In a particular embodiment the blood-derived sample is a plasma sample. Plasma (or blood plasma) is the liquid component of blood, as is known to the skilled person. Plasma may be obtained from whole blood by removal of blood cells from the blood. Appropriate methods for separation of blood cells and plasma (i.e. blood fractionation) are well known in the art. For instance, plasma may be obtained by centrifugation of whole blood, which is the current standard procedure in the art. To prevent clotting, an anticoagulant is added to the blood sample upon collection. Suitable anticoagulants are well known in the art and include sodium EDTA and potassium EDTA, sodium citrate, heparin and potassium oxalate. Appropriate centrifugation protocols are known in the art, for instance an exemplary centrifugation protocol would be to spin whole blood containing an anticoagulant at 1,300×g for 20 mins at 4° C. The upper phase of centrifuged whole blood is the plasma, which may be removed from the other phases using e.g. a pipette. The invention may thus comprise steps of adding an anticoagulant to a blood sample from the subject, and subsequently centrifuging the blood sample in order to isolate a plasma sample from the subject.

In another embodiment, the blood-derived sample is a serum sample. Serum (or blood serum) is blood plasma lacking clotting factors. Serum can also be considered the liquid fraction of blood remaining following clotting. Methods to isolate serum from blood are well known in the art. Generally, serum is obtained by collecting a whole blood sample, and allowing the sample to clot by leaving it undisturbed at room temperature, for e.g. 15-30 mins or as long as is required for a clot to form. Thus, unlike the process of plasma isolation, no anticoagulant is added to a blood sample if it is desired to isolate the serum.

The clotted blood is then centrifuged to separate the serum and the blood clot. A suitable centrifugation protocol is to spin the sample at about 1,500×g for 10 mins at 4° C. Following centrifugation the upper phase of the sample is serum, which may be removed from the other phases using e.g. a pipette. The invention may thus comprise steps of allowing a blood sample from the subject to clot, and subsequently centrifuging the blood sample in order to obtain a serum sample from the subject.

The concentrations of TRIM28, PLOD1 and CEACAM5 in the blood-derived sample are then determined. Optionally, the concentration of P4HA1 in the blood-derived sample is also determined. The concentrations of the proteins may be determined by any suitable method. Several such methods, i.e. methods by which the concentration of a protein in a sample can be determined, are known in the art.

In a particular embodiment, the concentrations of the proteins of interest are determined using an immunoassay. By the term immunoassay is meant any assay which utilises an antibody in detection/quantification of the protein of interest. Any antibody, as defined and described above, may be used.

Antibody fragments are discussed in Rodrigo et al., Antibodies, Vol. 4(3), p. 259-277, 2015. Antibody fragments include, for example, Fab, F(ab′)2, Fab′ and Fv fragments, all of which are well-known and understood in the art. A Fab fragment consists of the antigen-binding domain of an antibody, i.e. an individual antibody may be seen to contain two Fab fragments, each consisting of a light chain and its conjoined N-terminal section of the heavy chain. Thus a Fab fragment contains an entire light chain and the VH and C_{H}1 domains of the heavy chain to which it is bound. Fab fragments may be obtained by digesting an antibody with papain.

F(ab′)2 fragments consist of the two Fab fragments of an antibody, plus the hinge regions of the heavy chains, including the disulphide bonds linking the two heavy chains together. In other words, a F(ab′)2 fragment can be seen as two covalently joined Fab fragments. F(ab′)2 fragments may be obtained by digesting an antibody with pepsin. Reduction of F(ab′)2 fragments yields two Fab′ fragments, which can be seen as Fab fragments containing an additional sulfhydryl group which can be useful for conjugation of the fragment to other molecules.

Fv fragments consist of just the variable domains of the light and heavy chains. These are not covalently linked and are held together only weakly by non-covalent interactions. Fv fragments can be modified to produce a synthetic construct known as a single chain Fv (scFv) molecule. Such a modification is typically performed recombinantly, by engineering the antibody gene to produce a fusion protein in which a single polypeptide comprises both the V_{H} and V_{L} domains. scFv fragments generally include a peptide linker covalently joining the V_{H} and V_{L} regions, which contributes to the stability of the molecule. The linker may comprise from 1 to 20 amino acids, such as for example 1, 2, 3 or 4 amino acids, 5, 10 or 15 amino acids, or other intermediate numbers in the range 1 to 20 as convenient. The peptide linker may be formed from any generally convenient amino acid residues, such as glycine and/or serine. One example of a suitable linker is Gly₄Ser. Multimers of such linkers may be used, such as for example a dimer, a trimer, a tetramer or a pentamer, e.g. (Gly₄Ser)₂, (Gly₄Ser)₃, (Gly₄Ser)₄ or (Gly₄Ser)₅. However, it is not essential that a linker be present, and the V_{L} domain may be linked to the VH domain by a peptide bond.

Other types of antibody derivative molecules and constructs comprising antigen-domains of antibodies are known and described in the art, including for example, multimeric antibodies, chimeric antibodies, engineered or recombinant antibodies, minibodies, diabodies, and various others. Any such antibody, as appropriate, may be used in the immunoassay.

A preferred immunoassay for protein concentration determination is quantitative ELISA. Methods for performing quantitative ELISA are well known in the art. Moreover, kits for the performance of quantitative ELISA are commercially available and include instructions/protocols. A kit for performing quantitative ELISA to determine the concentration of TRIM28 in a sample is available from MyBiosource, Inc. (San Diego, USA), product no. MBS9902706; a kit for performing quantitative ELISA to determine the concentration of PLOD1 in a sample is available from Aviva Systems Biology Corp. (San Diego, USA), product no. OKDD00473; a kit for performing quantitative ELISA to determine the concentration of CEACAM5 in a sample is available from LifeSpan BioSciences, Inc. (Seattle, USA), product no. LS-F24454; and a kit for performing quantitative ELISA to determine the concentration of P4HA1 in a sample is available from Signalway Antibody LLC (Baltimore, USA), product no. EK2716.

Any form of ELISA assay may be used in the methods of the invention, including direct ELISA, indirect ELISA and sandwich ELISA. Sandwich ELISA is preferred for use in the invention, since only the antigen of interest is captured onto the ELISA plate during the course of the assay. Protocols for performing ELISA sandwich assays are well known in the art. According to an exemplary, non-limiting sandwich ELISA protocol, an antibody against the antigen of interest is first immobilised onto the wells of an ELISA plate (i.e. in the present invention antibodies against TRIM28, PLOD1 and CEACAM5, and optionally P4HA1, are immobilised onto the wells of ELISA plates. Antibodies against all these proteins are widely commercially available. By an antibody “against” a particular molecule is meant an antibody which specifically binds that molecule, e.g. an antibody against TRIM28 may alternatively be referred to as an antibody which specifically binds TRIM28 or which recognises TRIM28). The antibody immobilised to the wells of the ELISA plate is known as the “capture antibody”.

Antibodies may be immobilised onto ELISA plates using a variety of methods, for instance passive adsorption, whereby the antibody is applied to the plate in a coating buffer and the plate incubated. Any suitable coating buffer may be used. An example of a suitable coating buffer is carbonate/bicarbonate buffer, containing e.g. 0.2 M sodium carbonate/bicarbonate at about pH 9.5. The capture antibody may be contained in the coating buffer at a concentration in the range 1-10 μg/ml, and the adsorption procedure performed overnight at 4° C. Alternative methods for immobilisation of the capture antibody include e.g. affinity attachment or crosslinking of the antibody to the ELISA plate. For affinity attachment, an antibody comprising a tag is generally used as the capture antibody, so that the tag can bind its specific binding partner on the surface of the ELISA plate. Any immobilisation method known in the art may be used.

ELISA plates are well known in the art, including ELISA plates designed specifically for antibody attachment by adsorption, antibody attachment by affinity binding and antibody attachment by crosslinking. Such plates are available from e.g. Thermo Fisher Scientific (USA). All ELISA plates are multi-well plates. Common examples include 96-well and 384-well ELISA plates. For instance, ELISA plates designed for antibody attachment by adsorption commonly have hydrophilic surfaces; ELISA plates designed for antibody attachment by affinity binding are generally coated with a binding partner for a tag attached to the antibody, e.g. if the antibody has a polyhistidine (His) tag the ELISA plate may have surfaces coated with nickel, or if the antibody has a glutathione S-transferase (GST) tag the ELISA plate may have surfaces coated with glutathione. A suitable ELISA plate is chosen based on the intended method of capture antibody immobilisation.

As an alternative to an ELISA plate, an ELISA strip may be used, e.g. an 8-well or 10-well strip. An ELISA strip is essentially a smaller version of an ELISA plate useful for smaller assays using fewer samples.

Following immobilisation of the capture antibody, the wells of the plate may be washed, e.g. with PBS, and then blocked. Suitable blocking agents include e.g. PBS containing 1-5% w/v non-fat dry milk or 1-5% BSA. Blocking may be performed overnight at 4° C. Any suitable blocking protocol, utilising any suitable blocking agent, may be used. Following blocking, the plate may be washed again, e.g. with PBS.

Following application of the antibody to the plate and blocking, the sample (i.e. in this instance the blood-derived sample) is applied to the wells, and the plate incubated to allow binding of the target antigen to the adsorbed capture antibody. Serially diluted standards containing known concentrations of the protein of interest are added to separate wells of the ELISA plate. The sample (i.e. blood-derived sample) may also be applied in series dilution. The plate is then incubated, e.g. for 90 mins at 37° C.

Following sample incubation, the sample (and standards) are removed from the wells, and the plate may be washed again (e.g. with PBS). A detection antibody, which also binds the antigen of interest, is then applied to the wells and the plate incubated, e.g. for 2 hr at room temperature. The detection antibody may be conjugated to a detectable label to enable detection of the bound antibody. Alternatively, the detection antibody may be non-conjugated. Following application of the detection antibody, it is removed from the plate and the plate washed (e.g. with PBS). If the detection antibody is not conjugated to a detectable label, a secondary antibody which is conjugated to a detectable label is then applied, and the plate incubated (e.g. for 2 hr at room temperature). The secondary antibody is then removed and the plate may be washed again.

Alternatively, the detection antibody may be conjugated to biotin. Following application of the detection antibody, and washing of the plate, a labelled avidin (or streptavidin) conjugate is applied instead of a secondary antibody.

Suitable detectable labels are known in the art. Preferably the label enables colourimetric detection of antibody binding, such that binding of the antibody to the plate-bound sample can be detected using a plate reader. Examples of such labels include horseradish peroxidase (HRP) and alkaline phosphatase (ALP). HRP cleaves hydrogen peroxide, in a reaction coupled to oxidation of a hydrogen donor which undergoes a colour change during the reaction. Examples of suitable donors include 3,3′,5,5′-tetramethylbenzidine (TMB) and o-phenylenediamine dihydrochloride (OPD). To detect the colour change, the reaction is first stopped using a stopping solution (e.g. H₂SO₄) and the ELISA plate analysed in a plate reader. The analysis comprises measuring of the optical density of the solutions in the wells at an appropriate wavelength. If TMB is used, the wavelength is 450 nm; if OPD is used, the wavelength is 492 nm. ALP cleaves P-Nitrophenyl-phosphate (pNPP) in a chromogenic reaction which yields nitrophenol. The reaction can be stopped using NaOH, and the plate analysed using a plate reader by measuring optical density at 405 nm. Alternatively, detectable labels which enable chemifluorescent or chemiluminescent detection of the antigen may be used. Any detectable label known in the art may be used.

The optical densities of the series of standards are used to generate a standard curve correlating optical density with target molecule concentration. The concentration of the antigen of interest in the sample can then be calculated based on simple comparison to the standard curve.

In any sandwich ELISA assay, the combination of capture and detection antibodies must be selected to use a “matched pair”. This requires that the capture and detection antibody each recognise the target antigen at a separate epitope (or alternatively at an epitope which appears several times on the surface of the target antigen) such that binding of the capture antibody to the antigen does not block binding of the detection antibody to the antigen. Additionally, if a secondary antibody is used to detect the detection antibody, the detection antibody and capture antibody must have been raised in different species, to prevent binding of the secondary antibody to the capture antibody. Whether a particular pair of antibodies is suitable for use in combination can be determined empirically using standard procedures in the art. Use of a matched pair of antibodies can be ensured by using a commercial ELISA kit for the assay. Both capture and detection antibodies are preferably monoclonal.

By “monoclonal antibody” is meant an antibody preparation consisting of a single antibody species, i.e. all antibodies in the preparation have the same amino acid sequences and thus bind the same epitope on their target antigen. A monoclonal antibody may be obtained from a hybridoma, using techniques standard in the art.

In quantitative ELISA, e.g. following the protocol set out above, an antibody fragment may be used as the antibody for capture and/or detection. The protocol may be tailored to account for this. In particular, if an antibody fragment is used for antigen detection, it will not generally be possible to use a secondary anti-Fc antibody to bind the detection antibody fragment. Thus the detection antibody fragment would need either to comprise a detectable label itself, or to be tagged to allow secondary detection. For instance, as described above, the detection antibody fragment could be conjugated to biotin, to allow detection using an avidin or streptavidin conjugate comprising a detectable label. Alternatively the detection antibody fragment could contain a tag, such as a His tag, FLAG tag or HA tag, which can be bound by a secondary antibody which comprises a detectable label. Where an antibody fragment is used, preferably it is a fragment of a monoclonal antibody.

Another immunoassay by which protein concentration can be determined is quantitative Western blot. Quantitative Western blots can be performed using densitometry. A method for performing quantitative Western blotting is disclosed in Taylor & Posch, Biomed Research International 2014, Article ID 361590).

Once the concentrations of the proteins of interest (i.e. TRIM28, PLOD1 and CEACAM5, and optionally P4HA1) have been determined, a further determination is made as to whether the subject is suffering from colorectal cancer. This determination is made based on either the summed or relative concentrations of the proteins of interest in the sample. A “summed concentration” may be the total, combined concentration of all proteins of interest in the sample. Alternatively, the “summed concentration” of the proteins of interest may be determined by adding together the concentrations of certain proteins of interest and subtracting concentrations of others. In particular, the concentrations of the proteins of interest may be modified based on constant values (e.g. by multiplying or dividing the concentrations of the proteins of interest by the determined constant values). By “relative concentrations” is meant the relationships between the concentrations of the proteins to one another. For instance, the determination of whether a subject has colorectal cancer may be made based on the ratios between the concentrations of the various proteins of interest, or the absolute concentrations of the proteins of interest.

The skilled person will be aware that concentration can be measured as a molar value, or in the format weight/volume. Either form of concentration measurement can be used according to the methods of the invention, though as detailed below, in the specific embodiments of the invention disclosed herein the protein concentrations are measured in the format weight/volume (e.g. ng/ml and pg/ml).

The invention provides a particular embodiment in which the determination as to whether the subject is suffering from colorectal cancer is made based upon the concentrations of TRIM28, PLOD1 and CEACAM5, as follows:

i) if the concentration of TRIM28 is greater than 0.3 ng/ml, the subject does not have colorectal cancer;

ii) if the concentration of TRIM28 is less than or equal to 0.3 ng/ml and the concentration of PLOD1 is greater than or equal to 1.7 ng/ml, the subject has colorectal cancer;

iii) if the concentration of TRIM28 is less than or equal to 0.3 ng/m l, the concentration of PLOD1 is less than 1.7 ng/ml and the concentration of CEACAM5 is greater than or equal to 0.13 pg/ml, the subject has colorectal cancer; and

iv) if the concentration of TRIM28 is less than or equal to 0.3 ng/ml, the concentration of PLOD1 is less than 1.7 ng/ml and the concentration of CEACAM5 is less than 0.13 pg/ml, the subject does not have colorectal cancer.

In a further, more particular version of the same embodiment, the determination as to whether the subject is suffering from colorectal cancer is made as follows:

i) if the concentration of TRIM28 is greater than 0.27 ng/ml, the subject does not have colorectal cancer;

ii) if the concentration of TRIM28 is less than or equal to 0.27 ng/ml and the concentration of PLOD1 is greater than or equal to 1.69 ng/ml, the subject has colorectal cancer;

iii) if the concentration of TRIM28 is less than or equal to 0.27 ng/ml, the concentration of PLOD1 is less than 1.69 ng/ml and the concentration of CEACAM5 is greater than or equal to 0.125 pg/ml, the subject has colorectal cancer; and

iv) if the concentration of TRIM28 is less than or equal to 0.27 ng/ml, the concentration of PLOD1 is less than 1.69 ng/ml and the concentration of CEACAM5 is less than 0.125 pg/ml, the subject does not have colorectal cancer.

The invention provides another particular embodiment in which the determination as to whether the subject is suffering from colorectal cancer is made based upon the concentrations of TRIM28, PLOD1, CEACAM5 and P4HA1 as follows:

i) if (6.7×[TRIM28])−(0.7×[PLOD1])−(13.1×[CEACAM5])+(0.4×[P4HA1])−2.2 is less than zero, the subject has colorectal cancer; and

ii) if (6.7×[TRIM28])−(0.7×[PLOD1])−(13.1×[CEACAM5])+(0.4×[P4HA1])−2.2 is greater than or equal to zero, the subject does not have colorectal cancer.

In a further, more particular version of the same embodiment, the determination as to whether the subject is suffering from colorectal cancer is made as follows:

i) if (6.7×[TRIM28])−(0.65×[PLOD1])−(13.13×[CEACAM5])+(0.43×[P4HA1]) −2.21 is less than zero, the subject has colorectal cancer; and

ii) if (6.7×[TRIM28])−(0.65×[PLOD1])−(13.13×[CEACAM5])+(0.43×[P4HA1]) −2.21 is greater than or equal to zero, the subject does not have colorectal cancer.

In this embodiment, in line with standard annotation, [TRIM28] means the concentration of TRIM28; [PLOD1] means the concentration of PLOD1; [CEACAM5] means the concentration of CEACAM5; and [P4HA1] means the concentration of P4HA1. In this embodiment, the concentrations of TRIM28, PLOD1 and P4HA1 are each measured in ng/ml. The concentration of CEACAM5 is measured in pg/ml.

As mentioned above, the methods of the invention may conveniently be automated. In particular, the step of determining, based on the determined protein concentration values, whether the subject is suffering from colorectal cancer may be performed by a computer processor programmed to perform said step. In another embodiment the step of determining (e.g. calculating) the protein concentrations (e.g. deriving the protein concentrations from the values obtained from the assay used, i.e. from the assay output values), may alternatively, or additionally, be performed by a computer processor programmed to perform said steps. Further, the performance of the assay steps to measure the protein concentration may be automated. Thus, the assay may be performed in an apparatus comprising a computer processor programmed to control the apparatus to perform the assay.

The step of measuring, or of obtaining or calculating the protein concentrations may be performed by one computer processor and the step of determining whether the subject is suffering from colorectal cancer may be performed by a separate computer processor. Preferably, both steps are performed by a single computer processor programmed to perform both steps.

For instance, the step of measuring protein concentrations may be automated such that the blood-derived sample can be provided to a machine controlled by a computer processor programmed to perform the step of measuring the protein concentrations, such that once the sample is provided the concentration determination step is performed by the machine. For instance, the computer processor may be programmed such that it controls the performance of a quantitative ELISA by the machine. Machines for the performance of automated ELISAs are known in the art, including e.g. the ELISA NIMBUS (Hamilton Company, USA).

A computer processor may similarly be programmed to perform an analysis of the protein concentrations in order to determine whether the subject has colorectal cancer. For instance, a computer processor may be programmed to carry out the analyses described above to determine whether the subject has colorectal cancer, based on the determined protein concentrations. The computer processor may require that the relevant protein concentrations are input by a user. Alternatively, the relevant protein concentrations may be obtained directly from the measurement step. For instance, a single computer processor may direct a machine to perform a quantitative ELISA to measure the protein concentrations (upon provision of the blood-derived sample to the machine), and subsequently analyse the determined protein concentrations to determine whether the subject has colorectal cancer.

In another aspect, the invention provides a method of diagnosing and treating colorectal cancer in a subject. The diagnosis of colorectal cancer is performed as described above. Thus the diagnostic procedure includes a diagnostic method of the invention, but may further include additional diagnostic testing, in particular colonoscopy and possibly biopsy of the suspected cancer. As detailed above, the subject may be any human subject. Colorectal cancer is defined above.

In this method of the invention, if the subject is diagnosed with colorectal cancer, treatment for colorectal cancer is administered to the subject. Any treatment known in the art as suitable for treatment of colorectal cancer may be administered. For instance, the treatment may comprise surgery, to remove the cancer from the subject. The treatment may comprise radiotherapy, which may be administered externally (i.e. from outside of the body) or internally (i.e. brachytherapy), whereby a source of radiation is inserted into the anus and applied directly to the cancer. The treatment may comprise chemotherapy, which may be administered by any appropriate route (e.g. orally or intravenously). Suitable chemotherapy drugs for colorectal cancer include 5-fluorouracil (5FU), capecitabine, oxaliplatin (optionally in combination with 5FU or capecitabine) and irinotecan (optionally in combination with 5FU). The treatment may comprise immunotherapy, such as administration of a monoclonal antibody. For instance, a checkpoint inhibitor may be administered. Adoptive cell therapy may be used, for instance WO 2017/194555 discloses a T-cell receptor which may be used in therapy for CRC which displays microsatellite instability. Any combination of these therapies may be used, in accordance with the judgement of a skilled physician in light of the characteristics of the subject and the cancer.

Depending on the stage at which the cancer is diagnosed, the treatment administered may be curative (or intended to be curative) or palliative. Any cancer treatment may be administered, in accordance with standard medical practice.

Notably, the steps of diagnosing colorectal cancer and treating it may be, and indeed are likely to be, performed by different individuals. For instance, as described above, the blood sample may be obtained from the subject by a clinician, nurse or phlebotomist. The diagnostic procedure of the invention may be performed by e.g. a pathologist or clinical biochemist, potentially using an automated system as described above. The treatment may then be administered by a physician, e.g. a surgeon or oncologist.

In another aspect the invention provides a kit comprising a set of reagents for determining the presence or concentration of TRIM28, PLOD1 and CEACAM5 in a sample. The kit may further comprise a reagent for determining the presence or concentration of P4HA1 in a sample.

In a particular embodiment, the sample is a blood-derived sample as defined above. The reagents are preferably provided separately, each in its own, separate container within the kit. The kit may be suitable for use in the diagnostic methods described above. The kit may be provided in for example a box or package, and in addition to the reagents may contain a set of instructions for its use.

As noted above, a reagent for determining the presence or concentration of a given protein may be any reagent or set or combination of reagents which may be used specifically to detect that protein (i.e. to distinguish it from other proteins or molecules which may be present in the sample), and to determine the amount or level of protein present in the sample. The reagent(s) may thus be specific for a target protein and may report on its presence or absence, or amount, in the sample.

In a particular embodiment, the kit comprises:

i) a specific binding agent which binds TRIM28, or a fragment thereof;

ii) a specific binding agent which binds PLOD1, or a fragment thereof; and

iii) a specific binding agent which binds CEACAM5, or a fragment thereof.

“Specific binding agents” are defined above. The specific binding agents may bind a fragment, or part, of TRIM28, PLOD1 and CEACAM5, respectively. That is to say each specific binding agent does not require the full-length target protein for binding to take place. Rather, each agent may also specifically bind to fragments of its molecular partner. Clearly this does not mean that each agent will bind any and every fragment of its molecular partner. Rather, each specific binding agent will bind any fragment of its molecular partner which contains its binding site. Thus for instance if the specific binding agent is an antibody, it will bind any fragment of its antigen which contains its epitope.

The kit of the invention may further comprise a specific binding agent which binds P4HA1, or a fragment thereof.

In a particular embodiment of the invention, each specific binding agent in the kit is an antibody. Preferably, each antibody is a monoclonal antibody. In particular embodiments, the specific binding agent may be a fragment of a full-length antibody, preferably of a full-length monoclonal antibody, as discussed above.

The antibodies of the kit of the invention may be from any species. For instance, the antibodies may be human, or they may be non-human, e.g. they may be from mouse, rat, rabbit, goat or any other animal from which antibodies are commonly or may be obtained. The antibodies of the kit may all be from the same species, or may be from different species. The antibodies may be of any isotype, i.e. IgG, IgE, IgM, IgA or IgD, and may be of any sub-type within their isotype, e.g. if an antibody is an IgG antibody it may be of the IgG1, IgG2, IgG3 or IgG4 sub-type. The antibody may comprise a λ light chain or κ light chain. The antibodies in the kit may all be of the same isotype or may be of different isotypes.

Conveniently, the specific binding agent may be provided with a means for detecting the specific binding agent when it is bound to its target protein, particularly with means by which it may be distinguished from other specific binding proteins. Such means may be any reporter, that is any moiety or entity that may be used to provide, directly or indirectly, a signal which may be detected. Thus, the reporter may be directly or indirectly signal-giving. For example, the reporter may be a detectable label, for example a detectable label which can be directly detected, e.g. an optically or spectrophotomerically detectable label, e.g. a colorimetric or fluorescent label or such like, or a radioisotope, or quantum dot, nanoparticle or such like. Alternatively, the reporter may generate a signal through a further reaction, or in conjunction with further components, or it may provide a binding site for a detectable label, or for a further detection molecule itself comprising a detection label (e.g. for a labelled detection probe) or for further signal-generating means.

The kit of the invention may be for performing an ELISA, i.e. the kit may be an ELISA kit. The ELISA kit comprises a detection antibody for each antigen. The detection antibody may comprise a detectable label. Detectable labels are described above. In a representative embodiment, the detection antibody may alternatively comprise a covalently conjugated biotin molecule. The kit may further comprise a secondary antibody, which specifically binds the detection antibody. If the detection antibody is a biotin conjugate, the kit may comprise an avidin or streptavidin construct comprising a detectable label. In an alternative, the detection antibody may comprise an avidin or streptavidin tag, and the kit further comprise a biotin construct comprising a detectable label.

The ELISA kit may preferably be a sandwich ELISA kit. In this instance, the kit may further comprise a capture antibody for each antigen. Requirements for compatibility of capture antibodies and detection antibodies are described above. If a capture antibody is provided, the capture antibody and detection antibody provided for each antigen form a matched pair. The requirements for pairing apply equally regardless of whether the antibodies used are antibody fragments or full-length antibodies.

The ELISA kit may further include one or more ELISA plates. If the ELISA kit is a sandwich ELISA kit, the ELISA plates are preferably provided with the capture antibody pre-immobilised on the surfaces of the wells. The wells are preferably also pre-blocked. Alternatively, a blocking agent (e.g. BSA) may be provided in the kit.

The ELISA kit may further comprise a detection reagent, appropriate to the detectable label used on the detection or secondary antibodies. Suitable detection reagents for use with common detectable labels are described above.

The ELISA kit may further comprise purified TRIM28, PLOD1 and CEACAM5, and optionally P4HA1, for use in standards to generate a standard curve. These may be provided in series dilution, or alternatively in a stock solution to be serially diluted by the user. Purified versions of these proteins may be commercially available, e.g. recombinant TRIM28 can be purchased from Abcam (UK), product no. ab131899. Alternatively, the genes encoding these proteins can be cloned, and the proteins recombinantly expressed and purified, using standard techniques in the art.

The invention further provides the use of a kit as described above in the diagnosis of colorectal cancer, wherein said diagnosis comprises the performance of a method of the invention as described above. Similarly, the invention provides a method of diagnosing colorectal cancer comprising using a kit as described above to perform a diagnostic method as described above. Similarly, the invention provides a method of diagnosing and treating colorectal cancer, comprising using a kit as described above to perform a diagnostic method as described above, and, if the subject for whom the diagnostic method is performed is diagnosed with colorectal cancer, administering treatment for colorectal cancer to the subject, as described above. In particular, an ELISA kit of the invention may be used to perform a quantitative sandwich ELISA assay, as described above.

Also provided is a computer programme product comprising instructions that, when executed, will cause a processor to perform a method of the invention. The computer programme product may be provided in the form of a tangible computer readable storage medium storing the computer program product, such as a CD. Such computer programme products are described above.

The invention may be further understood by reference to the non-limiting examples below, and the figures in which:

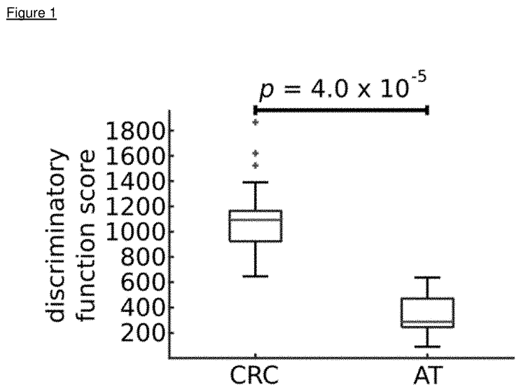

FIG. 1 shows the Classification accuracy of 22 paired samples: colorectal cancer tumour samples (CRC) and adjacent tissue (AT). The boxplot presents the discriminatory function score for CRC and AT groups (sum of the unit of parts per million [ppm] reported in Hao et al. (Sci Rep 7:42436, 2017)) for the selected proteins: PLOD1, P4HA1, LCN2, GNS, C12orf10, P3H1, TRIM28, CEACAM5, MAD1L1. p values were calculated using the double-sided Wilcoxon Signed Rank test. The bars in the boxes represent the median, 25^{th} and 75^{th} percentiles, while whiskers extend to ±2.7σ.

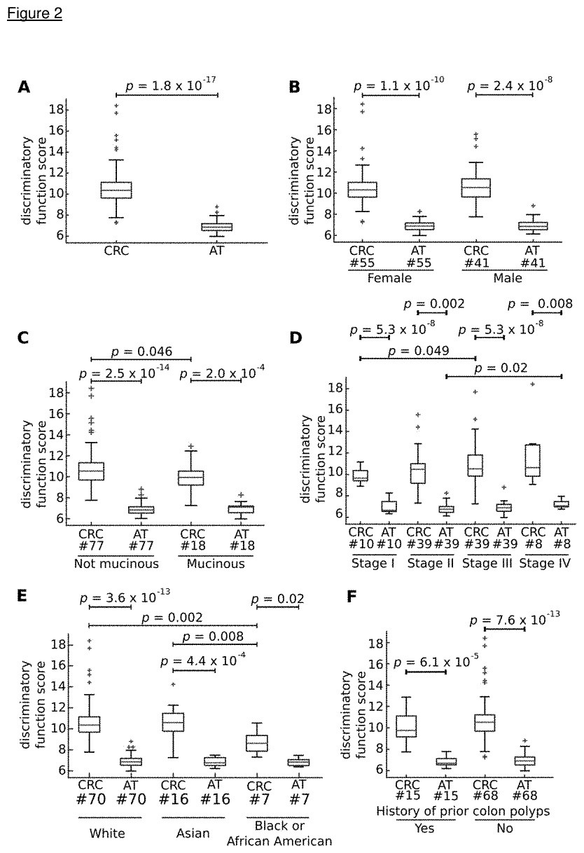

FIG. 2 shows the classification accuracy of 96 paired samples: colorectal cancer tumour samples (CRC) and adjacent tissue (AT). The boxplot presents discriminatory function scores for the CRC and AT groups (sum of the two to the power of the Unshared Log Ratio scores reported in the CPTAC study (see below) for the selected proteins: PLOD1, P4HA1, LCN2, GNS, C12orf10, P3H1, TRIM28, CEACAM5, MAD1L1). Significance p values were calculated using the double-sided Wilcoxon Signed Rank test for paired samples and the Wilcoxon Rank Sum test for unpaired samples. The bars in the boxes represent the median, 25th and 75th percentiles, while whiskers extend to ±2.7σ. Numbers below the boxplots denote the number of observations per category. (A) Discrimination between samples derived from tumours (CRC) and AT. (B) Discrimination between CRC and AT samples in males and females separately. (C) Discrimination between CRC and AT samples depending on histological subtype. (D) Discrimination between CRC and AT samples depending on tumour stage. (E) Discrimination between CRC and AT samples depending on race. (F) Discrimination between CRC and AT samples depending on prior colon polyp history.

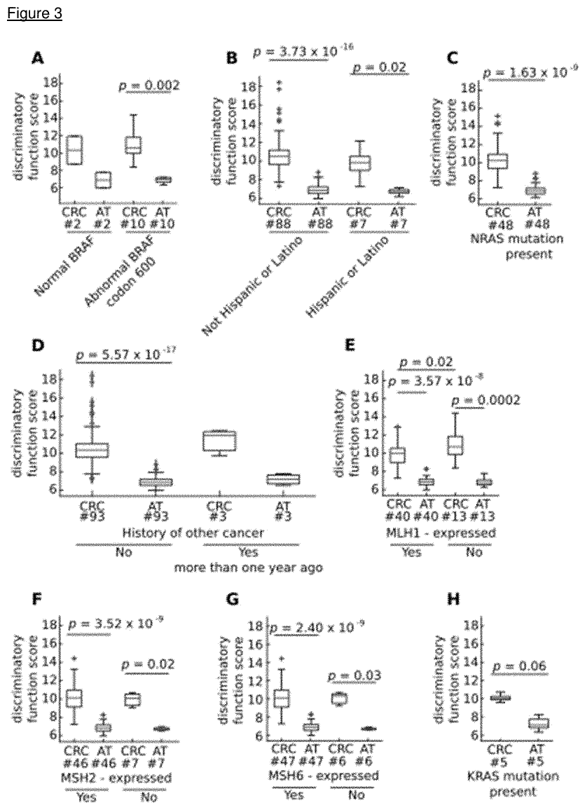

FIG. 3 shows the classification accuracy of colorectal cancer tumour samples (CRC) and adjacent tissue (AT). The boxplot presents discriminatory function score for the CRC and AT groups (sum of the 2 to the power of the Unshared Log Ratio scores reported in the CPTAC study for the selected proteins: PLOD1, P4HA1, LCN2, GNS, C12orf10, P3H1, TRIM28, CEACAM5, MAD1L1). The significance p value was calculated using the double-sided Wilcoxon Signed Rank test for paired samples and the Wilcoxon Rank Sum test for unpaired samples. The bars in the boxes represent the median, 25^{th} and 75^{th} percentiles, while whiskers extend to ±2.7σ. Numbers below the boxplots denote the number of observations per category. Discrimination between CRC and AT samples divided by (A) BRAF gene analyses results; (B) Ethnicity; (C) Presence of NRAS mutation (only one sample with no NRAS mutation is reported in the study); (D) History of other cancers; (E) MLH1 expression; (F) MSH2 expression; (G) MSH6 expression; (H) Presence of KRAS mutation (only one sample is reported in the study with no KRAS mutation).

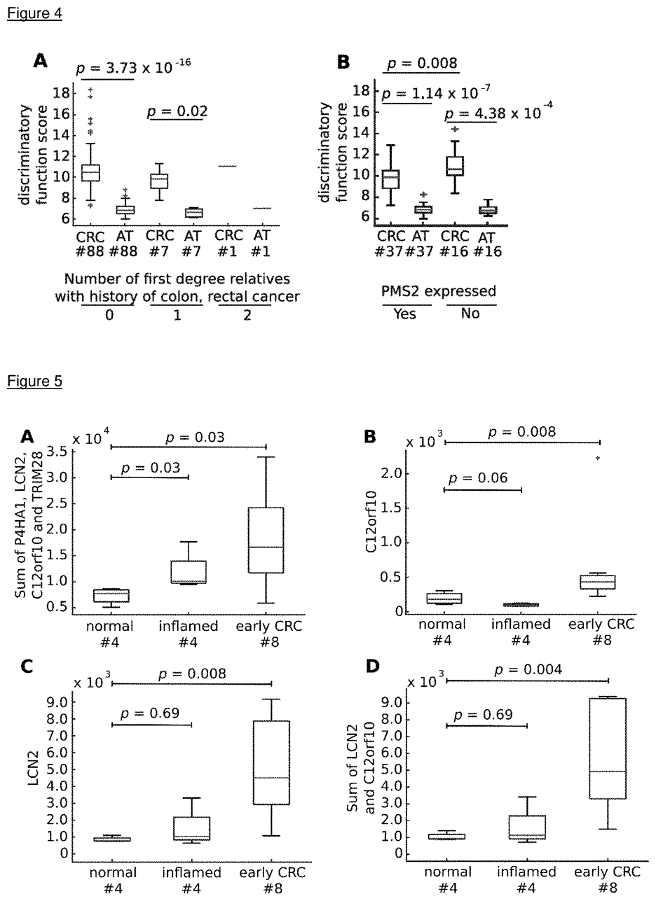

FIG. 4 shows the classification accuracy of colorectal cancer tumour samples (CRC) and adjacent tissue (AT). The boxplot presents discriminatory function scores for CRC and AT groups (sum of the 2 to the power of the Unshared Log Ratio scores reported in the CPTAC study for the selected proteins: PLOD1, P4HA1, LCN2, GNS, C12orf10, P3H1, TRIM28, CEACAM5, MAD1L1). The significance p value was calculated using the double-sided Wilcoxon Signed Rank test for paired samples and the Wilcoxon Rank Sum test for unpaired samples. The bars in the boxes represent the median, 25^{th} and 75^{th} percentiles, while whiskers extend to ±2.7σ. Numbers below the boxplots denote the number of observations per category. Discrimination between CRC and AT samples divided by (A) Number of first degree relatives with history of colorectal cancer; (B) PMS2 expression.

FIG. 5 shows the classification accuracy of 16 samples: early colorectal cancer tumour samples (CRC), normal and inflamed tissue. (A) Boxplot presenting the discriminatory function score (based on the sum of the four proteins present in the dataset out of the nine of interest: P4HA1, LCN2, C12orf10 & TRIM28). (B) C12orf10 expression differences in normal, inflamed and early CRC tissues. (C) LCN2 expression differences in normal, inflamed and early CRC tissues. (D) The sum of LCN2 and C12orf10 protein concentrations separates normal and early CRC tissue samples. Significance p values were calculated using the double-sided Wilcoxon Rank Sum test. The bars in the boxes represent the median, 25^{th} and 75^{th} percentiles, while whiskers extend to ±2.7σ. Numbers below the boxplots denote the number of observations per category.

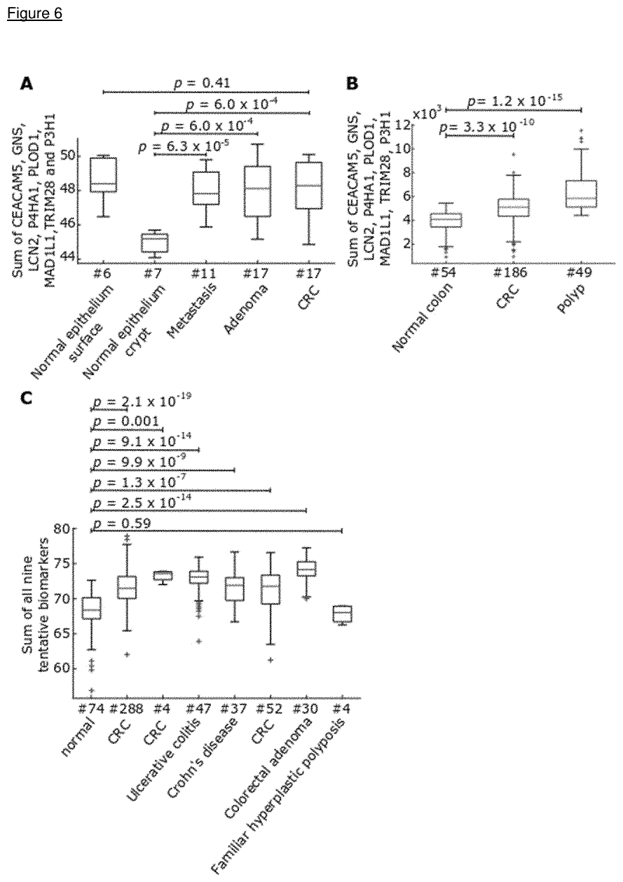

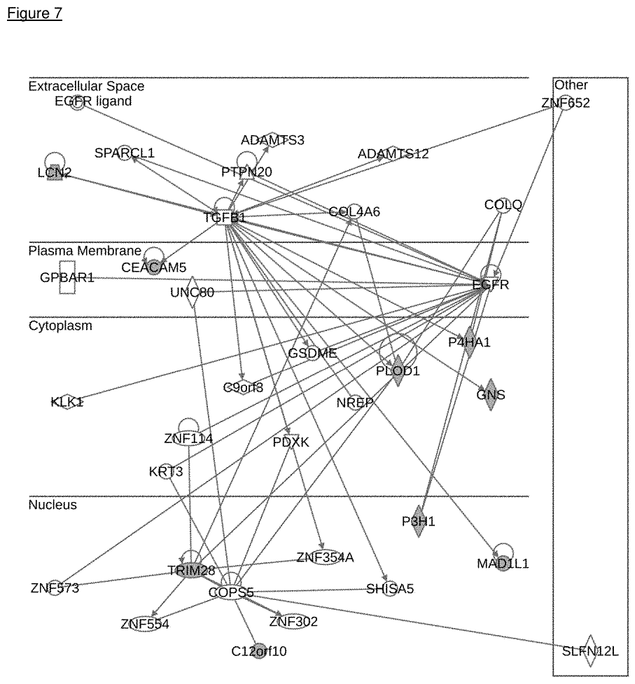

FIG. 6 shows the classification accuracy of colorectal tumour (CRC) samples and adjacent tissue (AT) in transcriptomic profiling datasets. (A) Transcriptome profiling of colorectal samples from 6 normal surface epithelium samples, 7 normal crypt epithelium samples, 17 CRC samples, 11 metastases and 17 adenomas (in total 19 subjects). (B) 54 normal colon tissue samples, 186 CRC samples and 49 polyps. (C) 74 normal samples, CRC samples from three different studies (n=4, 288 and 52, respectively), 30 adenomas, 4 familial hyperplastic polyposis samples, 47 ulcerative colitis samples and 37 Crohn's disease samples. For each plot significance p values were calculated using the double-sided Wilcoxon Rank Sum test. The bars in the boxes represent the median, 25^{th} and 75^{th} percentiles, while whiskers extend to ±2.7σ.

FIG. 7 shows based on Ingenuity Pathway Analysis that the nine selected proteins are highly interconnected and form a network module that regulates cell death and proliferation.

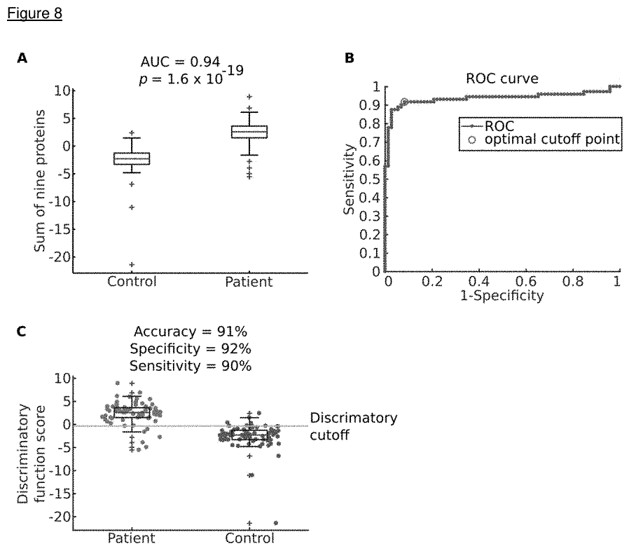

FIG. 8 shows the classification accuracy of 72 patients and 72 control samples in the training group. (A) Boxplot presenting the discriminatory function score based on protein concentrations in plasma samples measured with ELISA. The discriminatory function score was calculated as the sum of the nine proteins. The bars in the boxes represent the median, 25^{th} and 75^{th} percentiles, while whiskers extend to ±2.7σ. (B) Receiver operating characteristic curve (Kinsella et al., Database 2011:bar030) obtained for the classifier based on the sum of the concentrations of the nine proteins measured by ELISA in plasma samples of patients and controls. The optimal operating point (discriminatory function cut-off value) is marked with a circle, and it corresponds to the score value of −0.34. (C) Boxplot showing the discriminatory function score for patients and controls in the training set. The selected discriminatory function cut-off value is presented with a solid, horizontal line. Each dot represents an individual sample. The bars in the boxes represent the median, 25^{th} and 75^{th} percentiles, while whiskers extend to ±2.7σ.

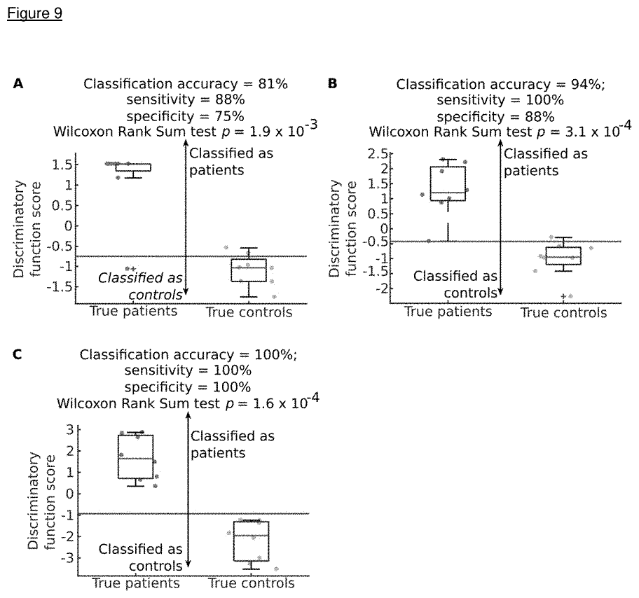

FIG. 9 shows the classification accuracy of different combinations of two, three or four proteins in a test set of 8 patients and control samples. The boxplots present discriminatory function scores based on the concentrations of combinations of two, three or four proteins in plasma samples of patients and controls in the test group, measured with ELISA. The results of the following protein combinations are shown: (A) TRIM28 and PLOD1; (B) TRIM28, PLOD1 and CEACAM5; and (C) TRIM28, PLOD1, CEACAM5 and P4HA1. The discriminatory function score was calculated as the sum of the concentrations of the respective proteins. The discriminatory cut-off value is indicated with a solid horizontal line. The dots denote values obtained for patients and controls in the test group respectively. The bars in the boxes represent the median, 25^{th} and 75^{th} percentiles, while whiskers extend to ±2.7σ. For the combination of two proteins the best results were obtained when summing the concentrations of TRIM28 and PLOD1 (sensitivity of 88%, specificity of 75%). The best combination of three proteins yielded sensitivity of 100% and specificity of 88% (sum of TRIM28, PLOD1, and CEACAM5). However, the best sample separation was possible when using four proteins, namely TRIM28, PLOD1, CEACAM5, and P4HA1 (both sensitivity and specificity of 100%).

### EXAMPLES

Protein biomarkers for CRC that can be measured in blood were identified by a process based on meta-analysis of published genome- and proteome-wide analyses of CRC tissues and adjacent tissues (AT). For the published analyses see Hao et al. (Sci Rep 7:42436, 2017), Shiromizu et al. (supra), Quesada-Calvo et al. (Clin Proteomics 14:9, 2017) and Torrente et al. (PLoS ONE 11(6): e0157484, 2016).

The inventors focused on differentially expressed genes whose protein products were potentially released extracellularly, according to the Human Protein Atlas (Petryszak et al., Nucleic Acids Res 44:D746-D752, 2016). In order to identify optimal combinations of a limited number of proteins (and diagnostic methods based on these), the inventors used their classification algorithm (Hellberg et al., Cell Reports 16:2928-2939, 2016). Finally, the inventors tested the identified biomarkers using plasma from patients with newly diagnosed CRC and healthy controls.

Materials & Methods

Protein Prioritisation Using Randomised Elastic Net

In order to rank proteins the inventors analysed proteome profiling data of 22 CRC patients—paired samples taken from tumour and AT (Hao et al., supra). For each identified protein fold-change was calculated as the average protein expression in colorectal tumour samples divided by the average protein expression in AT. Differential expression was obtained from Hao et al. (supra), analysed by paired t-test. For biomarker prioritisation using random elastic net the inventors pre-selected those proteins that were: a) differentially expressed (adjusted for multiple correction using procedure described by Storey) with a p value <0.01; b) upregulated in CRC tumour samples (fold change more than 2) and c) predicted to be secreted according to the Human Protein Atlas (https://www.proteinatlas.org, data downloaded in July 2017).

113 proteins fulfilling the above criteria were rank-ordered by their predictive value using randomised elastic net. Randomised elastic net was implemented as a modification of randomised lasso as described by Meinshausen et al. (Journal of the Royal Statistical Society: Series B (Statistical Methodology) 72: 417-473, 2010). Here, lasso was replaced with elastic net. For selected λ in cross-validation and for α=0.5, the inventors permuted data by adding random penalty factors for each predictor (protein) from the interval [1/α, 1]. Next, model coefficients were estimated (elastic net). 100,000 permutations were performed. Predictors with non-zero coefficients in at least one of the 100,000 permutations were selected. For downstream analyses proteins selected in at least 45% of permutations were chosen (corresponding to 9 proteins).

Sample Classification

Based on the selected proteins the inventors built a classifier with discriminative function being a sum of expression of all nine proteins. All zeros in proteomic data were replaced with NaN and treated as missing values (nansum( ) was used). To calculate Area Under ROC curve—AUC values—MATLAB function perfcurve( ) was used, having colorectal tumour (CRC) samples as the positive group and adjacent tissue (AT) samples as the negative group. In order to obtain significance values, the inventors performed the Wilcoxon Signed Rank test for paired samples and the Wilcoxon Rank Sum test for non-paired samples on the scores calculated based on the discriminative function score for both CRC and AT groups.

In order to test if selected proteins were better CRC biomarkers than would be obtained by chance, the inventors randomly selected nine upregulated proteins from the dataset and repeated the calculation of AUC scores 10,000 times. The permutation p value was calculated by comparing random AUC values with the original one.

Validation in Independent Datasets

To test the selected nine proteins, the inventors repeated the classification and classification tests as described above in two independent, publicly available proteomics datasets:
1) A set obtained from 101 individuals (Clinical Proteomic Tumor Analysis Consortium, CPTAC). The set contains samples taken from tumour sites (CRC) and adjacent tissue (AT). For classification the inventors used 2 to the power of the reported “Unshared Log Ratio” score. Unpaired samples were removed from the analyses;
2) A set obtained from four normal mucosa samples, four inflamed mucosa samples and eight early cancer samples (ProteomeXchange Dataset PXD005735). Additionally, the inventors analysed whether the transcriptome profiling of the nine genes encoding the selected proteins could discriminate between CRC and AT. The inventors analysed the following datasets:
1) EGEOD-77955: 6 normal surface epithelium, 7 normal crypt epithelium, 17 CRC, 11 metastases, 17 adenoma samples (In total 19 subjects);
2) E-GEOD-41258: 54 normal colon tissues, 186 CRC, 49 polyp samples;
3) E-MTAB-3732, which aggregated and normalised microarray datasets from different studies of healthy and diseased colorectal tissues. This included 74 healthy colon tissues, CRC from three studies with 4, 288 and 52 patients, 30 adenomas, 4 familial hyperplastic polyposis and 47 ulcerative colitis.

Discriminative Score and Clinical Data

In the publicly available dataset consisting of 100 individuals (CPTAC) the inventors also tested if the discriminative score was influenced by sex, histological subtype, history of prior polyps or race. Analysis was performed using the Wilcoxon signed rank test where samples were paired and the Wilcoxon rank sum test otherwise between specific subgroups of samples.

ELISA of Plasma Samples from Patients with CRC and Healthy Controls 80 CRC patients (40 females and 40 males, mean age of 71.8 years (range 34-89)) from south-eastern Sweden, who had undergone surgical resections for primary CRC at the Department of Surgery, Division of Surgical Care, Region Jönköping County, Jönköping, Sweden, were recruited. The CRC patients had tumours localized in the colon (n=37) or rectum (n=43) with TNM stages I-IV (I=13, II=34, III=29 and IV=4). The control group consisted of 80 healthy blood donors (40 females and 40 males, mean age of 55.9 years (range 33-67)) with no known history of CRC and from the same geographical region as the cancer patients.

Venous blood samples were collected and centrifuged within 1 hour of collection to separate plasma and blood cells. Plasma samples were stored at −80° C. in the Biobank of Laboratory Services, registration number 868, Region Jönköping County, Jönköping, Sweden until analysis. The study was reviewed and approved by the Regional Ethical Review Board in Linköping, Linköping, Sweden (98113 and 2013/271-31). All patients included in this study gave an informed written consent for utilisation of their material in research. Plasma levels of the nine potential biomarkers were analysed using commercial Enzyme-Linked Immunosorbent Assays (ELISAs) according to the manufacturer's instructions: C12orf10 (MyBiosource, Inc., San Diego, Calif., United States), CEACAM5 (LifeSpan BioSciences, Inc., Seattle, Wash., United States), GNS (MyBiosource, Inc.), LCN2 (Aviva Systems Biology Corp., San Diego, Calif., United States), MAD1L1 (Abbexa Ltd., Cambridge, United Kingdom), P3H1 (Abbexa Ltd.), P4HA1 (Signalway Antibody LLC, Baltimore, Md., United States), PLOD1 (Aviva Systems Biology Corp.), and TRIM28 (MyBiosource, Inc.). Protein levels of the nine potential biomarkers were determined using the Sunrise Tecan Microplate reader (Tecan Austria GmbH, Salzburg, Austria) along with the Magellan 7.x 2010 software (Tecan Austria GmbH). Protein levels of C12orf10, GNS, LCN2, MAD1L1, P4HA1, PLOD1 and TRIM28 were expressed as nanograms per millilitre (ng/mL). Protein levels of CEACAM5 and P3H1 were expressed as picograms per millilitre (pg/mL). In case protein values were out of the ELISA kit detection limit, for calculations we assumed either the ELISA kit maximum detection limit or a value of 0, as appropriate.

Network Analysis of the Identified Nine Proteins

The Ingenuity Pathway Analysis (IPA) software (Qiagen, Hilden, Germany) was used to test if the nine proteins were part of the same network module. Sub-network formation by IPA was performed as follows: first, all genes having direct or indirect interactions with the nine proteins served as “seeds” to generate networks. Such focus genes were combined into networks to maximise their specific interconnectivity (the connectivity between focus genes in comparison to the number of their interactions with other genes within the IPA global network). Additional genes from the IPA global network were added in order to connect smaller networks formed by the focus genes. Finally, the resulting networks were scored based on the number of focus genes they contained—the higher score the lower the probability of finding this number of focus genes within a given network by random chance.

Sample Separation into Training and Testing Sets

For each of the nine individual proteins, the inventors tested whether their expression differed significantly between patients and controls using the double-sided Wilcoxon Rank Sum test. Next, the inventors summed the expression of all nine proteins in order to obtain a discriminatory function score and calculated AUC (as before the MATLAB perfcurve( ) function was used for that purpose). Even though based on the tissue samples (proteome profiling) all nine proteins should be upregulated in patient samples, in the plasma samples some of the proteins were in fact downregulated in patients compared to controls. Therefore, concentrations of those proteins were subtracted from the discriminatory function score instead of being summed. The P value was calculated using the double-sided Wilcoxon Rank Sum test. Next, the inventors aimed to reduce the number of proteins necessary for the classifier to perform well. For these analyses, ten percent of patient samples (n=8) and ten percent of control samples (n=8) were randomly selected as a test group and excluded from the initial analyses, in which the inventors tried different combinations of the measured proteins. The optimal operating point (discriminatory cutoff value) was selected using MATLAB function perfcurve( ). This is based on moving a straight line from the point (0,1) with a slope defined as:

S=Cost(FP)−Cost ADDIN EN.CITE ADDIN EN.CITE.DATA 2/Cost ADDIN EN.CITE ADDIN EN.CITE.DATA 21−Cost ADDIN EN.CITE ADDIN EN.CITE.DATA 2·TN+FP/TP+FN

that crosses the ROC curve. Here TP, FP, TN, and FN are the true positives, false positives, true negatives and false negatives respectively, and classification cost is denoted as Cost( ) Since it is most important to reduce probability of false negatives (classification of patients as healthy controls) the inventors assumed zero cost for classification of TN and TP, 0.8 cost for misclassification of positive class, and 0.2 for misclassification of negative class.

Classification of the Test Group

Finally, using the classifier constructed as described above, the inventors classified the excluded samples of patients and controls, calculated accuracy, sensitivity and specificity, and p value (p value was calculated based on the discriminatory function score obtained for the excluded samples between patients and controls using the double-sided Wilcoxon Rank Sum test).

Results

Biomarker Selection

In order to rank proteins as putative biomarkers of CRC the inventors analysed publicly available proteome profiling of CRC samples and paired AT samples collected from 22 subjects (Hao et al., supra). First, the inventors preselected proteins that were:
a) differentially expressed (corrected for multiple testing, defined based on having paired t-test p value <0.01);
b) upregulated in CRC samples compared to AT samples (fold change >2); and
c) predicted by the Human Protein Atlas to be secreted.

The inventors identified 113 such proteins. Using random elastic net (Materials and Methods) the inventors ranked the proteins based on their predictive value to discriminate CRC from AT. For further analyses the inventors selected the top nine proteins: PLOD1 (Q02809), P4HA1 (P13674), LCN2 (P80188), GNS (P15586), C12orf10 (Q9HB07), P3H1 (Q32P28), TRIM28 (Q13263), CEACAM5 (P06731) and MAD1L1 (Q9Y6D9); (randomized elastic net frequency >0.45; Materials and Methods). The Inventors found that the sum of the expression values of those nine proteins discriminated between CRC and AT with high accuracy (Area Under receiver operating characteristic Curve AUC=1, Wilcoxon Signed Rank test p=4.0×10⁻⁵; FIG. 1). This was significantly higher than when using nine random proteins (permutation test p<1.0×10⁻⁴, OR=1.66). Out of those nine proteins, three were reported before as possible CRC biomarkers; namely CEACAM5 (commonly known as CEA), LCN2 and TRIM28 (Shiromizu et al., supra).

Biomarker Tests in Independent Proteomic Datasets

In order to assess the reproducibility of our results the inventors analysed two independent publicly available proteome profiling datasets of CRC. First, the inventors analysed a dataset consisting of 101 individuals—that data was generated by the National Cancer Institute Clinical Proteomic Tumor Analysis Consortium (CPTAC). The data consists of 96 paired samples obtained from tumour sites (CRC) and AT. Secondly, the inventors validated that the selected nine proteins could separate tumour samples from AT. The inventors obtained a nearly perfect classification accuracy (AUC=0.99, Wilcoxon Signed Rank test p=1.8×10⁻¹⁷, FIG. 2A), which was higher than obtained by chance for nine randomly selected proteins (permutation test p=1.0×10⁻⁴, OR=2.21).

In the CPTAC study, the authors report clinical data including sex, race, histological subtype and history of prior colon polyps. The inventors therefore tested whether any of those covariates had an impact on the sample classification (FIG. 2B-F, FIG. 3 and FIG. 4). The inventors found significant differences in the discriminatory function score only between mucinous and non-mucinous tumours (Wilcoxon Rank Sum test p=0.046; FIG. 2C); pathological tumour stage I and III (double-sided Wilcoxon Rank sum test p=0.049; FIG. 2D); AT samples from patients with tumour stage II and IV (Wilcoxon Rank sum test p=0.02, FIG. 2D); between races—black/African-American patients versus Asian or Caucasian, p value=0.008, and 0.002 respectively, FIG. 2E); expression or no-expression of MHL1 and PMS2 (Wilcoxon Rank Sum test p=0.02, FIG. 3E; and p=0.008, FIG. 4B respectively). Furthermore, the inventors tested the correlation between pathological tumour stage and discriminatory function score and found no significant correlation (Pearson PCC=0.12, p=0.12). However, as shown in FIG. 2 those covariates did not have a significant impact on overall classification accuracy.

The inventors then tested yet another proteomic dataset consisting of 76 tissue samples, in which four to five patient sample digests were pooled. In total, proteomics analyses were performed on eight pools from colorectal tissue samples obtained from early stages of CRC, eight pools of apparently normal tissue (at surgical margin) samples and four pools of inflamed mucosa samples (Quesada-Calvo et al., supra). This dataset contains only four out of the nine selected putative biomarkers: P4HA1 (P13674), LCN2 (P80188), C12orf10 (Q9HB07) and TRIM28 (Q13263). For this reason, a new classifier was created based on the sum of those four tentative CRC biomarkers, which yielded a high classification accuracy for discriminating early CRC from normal tissue (AUC=0.91, Wilcoxon Rank Sum test, unpaired samples, p=0.03, FIG. 5A). The inventors also found a significant difference between normal and inflamed tissue (Wilcoxon Rank Sum test p=0.03, FIG. 5A). However, the combination of the four proteins did not yield a higher AUC score than expected by chance for four random proteins (p=0.08, OR=1.68). Therefore, the inventors asked if individual proteins could discriminate normal from tumour tissues. The inventors found that LCN2 and C12orf10 differentiated between the two conditions (AUC=0.97, Wilcoxon Rank Sum test p=0.008 for both proteins), which was higher than expected by chance for a random single gene classifier (permutation test p=0.03, OR=1.86 for both proteins; FIG. 5B, C). A combination of those two proteins gave even higher classification accuracy (AUC=1, Wilcoxon Rank Sum test p=0.004; FIG. 5D), which is higher than expected for two random proteins (permutation test p=1.0×10⁻⁴, OR=1.9). This suggested that a subset of the nine proteins might be sufficient for a highly accurate classification of patients and controls.

Biomarker Tests in Independent Transcriptomic Datasets

The inventors also tested 3 transcriptome profiling studies of CRC. In case some of the selected nine genes were not expressed in the tested dataset, classifiers were built using genes that were expressed. Firstly, the inventors analysed a dataset consisting of 6 normal surface epithelium samples, 7 normal crypt epithelium samples, 17 CRC samples, 11 metastases and 17 adenoma samples (in total 19 subjects; EGEOD-77955). This dataset lacked details of expression of the C12orf10 gene. Therefore, the inventors created a classifier based on the remaining eight genes. The inventors obtained a high classification accuracy when comparing normal crypt epithelium samples to CRC, metastases and adenoma samples (AUC>0.95, Wilcoxon Rank Sum test p<6.1×10⁻⁴; FIG. 6A; Table 1). However, in comparison to normal surface samples good separation of the groups was not obtained (AUC<0.43, Wilcoxon Rank Sum test p>0.25; FIG. 6A; Table 1).

TABLE 1

| | Metastasis | Adenoma | CRC |
|---|---|---|---|
| versus normal surface epithelium | | | |
| AUC | 0.32 | 0.42 | 0.41 |
| P | 0.26 | 0.60 | 0.55 |
| versus normal crypt epithelium | | | |
| AUC | 1.00 | 0.96 | 0.96 |
| P | 6.3 × 10⁻⁵ | 6.0 × 10⁻⁴ | 6.0 x 10⁻⁴ |

Classification area under the receiver operating characteristic (Price etal., Nat Biotechnol 35: 747-756, 2017) of 6 normal surface epithelium samples and 7 normal crypt epithelium samples versus 17 CRC, 11 metastasis and 17 adenoma samples, respectively. Significance was calculated using the double-sided Wilcoxon Rank Sum test (p).

Similarly, in another transcriptome profiling study tested (E-GEOD-41258) the inventors obtained good separation between groups (AUC>0.77, Wilcoxon Rank Sum test p<3.4×10⁻¹⁰; FIG. 6B; Table 2). Here, the inventors compared 54 normal colon tissue samples with 186 primary CRC and 49 polyp samples. This dataset also lacked expression profiles of C12orf10, and therefore the classifier was made using the remaining eight genes.

TABLE 2

| | Primary CRC | Polyp |
|---|---|---|
| AUC | 0.78 | 0.96 |
| P | 3.3 × 10⁻¹⁰ | 1.2 x 10⁻¹⁰ |

Classification area under the receiver operating characteristic (Price etal., supra) of 54 normal colon tissues versus 186 primary CRC and 49 polyp samples, respectively. Significance was calculated using the double-sided Wilcoxon Rank Sum test (p).

Finally, the inventors analysed a dataset (E-MTAB-3732) that aggregated and normalised microarray data from colorectal samples from 74 healthy tissues, three studies of CRC (n=4, 288 and 52 respectively), 4 familial hyperplastic polyposis samples, 30 colorectal adenomas, 47 ulcerative colitis samples and 37 Crohn's disease samples. All nine tentative biomarkers were profiled. In this dataset, normal colorectal tissue samples were compared to other groups. This yielded high separation for most comparisons (AUC>0.82; Wilcoxon Rank Sum test p<0.002; FIG. 6C; Table 3). The inventors also found significant differences between normal tissue compared to CRC (moderate separation AUC=0.78, Wilcoxon Rank Sum test p=1.3×10⁻⁷). However, the classifier didn't differentiate between normal colorectal tissue and familial hyperplastic polyposis (AUC=0.42, Wilcoxon Rank Sum test p=0.59).

TABLE 2
Classification area under the receiver operating characteristic (Price et al., supra) of 74
normal samples versus 37 Crohn's disease samples, three CRC studies (n = 4 colon tumour, 288
colorectal adenocarcinoma and 52 colorectal carcinoma, respectively), 30 colorectal adenoma, 4
familiar hyperplastic polyposis samples and 47 ulcerative colitis samples. Significance was calculated
using the double-sided Wilcoxon Rank Sum test (p).

| | | | | | | Familial | |
|---|---|---|---|---|---|---|---|
| | Crohn's | CRC | CRC | Colorectal | CRC | hyperplastic | Ulcerative |
| | disease | (study 1) | (study 2) | adenoma | (study 3) | polyposis | colitis |
| AUC | 0.83 | 0.99 | 0.84 | 0.98 | 0.78 | 0.42 | 0.90 |
| p | 9.9 × 10⁻⁹ | 0.001 | 2.1 × 10⁻¹⁹ | 2.5 × 10⁻¹⁴ | 1.3 × 10⁻⁷ | 0.59 | 9.1 × 10⁻¹⁴ |

In summary, analyses of transcriptome profiling data indicated that the chosen nine tentative CRC biomarkers could separate colorectal tumour samples from apparently normal samples.

Network Analysis Supports Pathogenic Relevance of the Nine Proteins

In order to test the pathogenic relevance of the biomarkers the inventors performed Ingenuity Pathway Analysis, as previously described (Gustafsson et al., Science Translational Medicine 7(313):313ra178, 2015). Briefly, the background to the method is that proteins which are associated with the same disease tend to be functionally related and interact, forming network modules (Tan et al., Curr Colorect Canc R 12: 151-161, 2016). Thus, if the proteins did interact, this would support their pathogenic and biomarker relevance. Indeed, the inventors found that the nine proteins did form a network module, which had an overriding function, namely regulating cell death and proliferation (FIG. 7).

Analyses of Nine Potential Protein Biomarkers in Plasma Samples from CRC Patients and Healthy Controls

The inventors proceeded to test all nine selected biomarkers in plasma samples obtained from 80 patients with CRC and 80 controls. It was found that seven of the proteins differed significantly between patients and controls: PLOD1, median 10 (0-10) vs. 0.19 (0-7.9) ng/mL, p=1.19×10⁻²¹; LCN2, 2.0 (0-10) vs. 2.3 (1.3-4.2) ng/mL, p=4.84×10⁻²; MAD1L1, 0 (0-3.1) vs. 0 (0-10) ng/mL, p=2.73×10⁻²; CEACAM5, 0 (0-0.45) vs. 0 (0-3.8) pg/mL, p=1.52×10⁻³; P4HA1, 3.7 (0-17) vs. 5.9 (1.4-22) ng/mL, p=4.88×10⁻⁶; TRIM28, 0 (0-0.82) vs. 1.14 (0-20) ng/mL, p=1.00×10⁻²⁷; and GNS, 7.1 (2.8-13) vs. 10 (5.3-14) ng/mL, p=1.05×10⁻¹⁴. By contrast, no significant differences were found for C12orf10, 1.6 (0.1-11) vs. 1.6 (0.58-20) pg/mL, p=4.07×10⁻¹ and P3H1, 7.8 (0-86) vs. 5.9 (0-586) pg/mL, p=1.28×10⁻¹ (double-sided Wilcoxon Rank Sum test). Although seven of the proteins differed significantly between patients and controls, they showed considerable variability. This indicated that on their own, none of the proteins would suffice as potential biomarkers for early diagnosis. A combination of all nine proteins was then tested to determine whether this combination separated patients and controls with high accuracy. For that purpose, the inventors calculated AUC and p values as before (double-sided Wilcoxon Rank Sum test p=1.62×10⁻¹⁹; FIG. 8). While the p value was highly significant, there was some overlap between patients and controls, resulting in a specificity of 92% and a sensitivity of 90%.

Combinations of smaller numbers of proteins were then tested. Firstly, all possible combinations of the nine proteins were tested in a training set consisting of randomly selected 72 patients and 72 controls. As detailed above, optimal combinations of proteins, and diagnostic methods based on these, were determined using the inventors' classification algorithm. Based on the training set, the optimal combination of two proteins utilised TRIM28 and PLOD1; the optimal combination of three proteins utilised TRIM28, PLOD1 and CEACAM5; and the optimal combination of four proteins utilised TRIM28, PLOD1, CEACAM5 and P4HA1. The identified combinations were then tested in a test set consisting of the remaining 8 patients and 8 controls (FIG. 9). As shown, the combination of two proteins yielded a classification accuracy of 81% (sensitivity of 88%, specificity of 75%). The combination of three proteins was superior, with a classification accuracy of 94% (sensitivity 100%, specificity 88%), while the combination of four proteins displayed 100% accuracy. The combinations of three and four proteins, in particular, offer significant improvements to CRC screening accuracy relative to existing methods. Importantly, both display 100% sensitivity, indicating that false negatives can be minimised.

The diagnostic methodologies applied by the classification algorithms were determined. For the method based on three proteins, it was determined that the following diagnostic method was performed:

i) if the concentration of TRIM28 is greater than 0.27 ng/ml, the subject does not have colorectal cancer;

ii) if the concentration of TRIM28 is less than or equal to 0.27 ng/ml and the concentration of PLOD1 is greater than or equal to 1.69 ng/ml, the subject has colorectal cancer;

iii) if the concentration of TRIM28 is less than or equal to 0.27 ng/ml, the concentration of PLOD1 is less than 1.69 ng/ml and the concentration of CEACAM5 is greater than or equal to 0.125 pg/ml, the subject has colorectal cancer; and

iv) if the concentration of TRIM28 is less than or equal to 0.27 ng/ml, the concentration of PLOD1 is less than 1.69 ng/ml and the concentration of CEACAM5 is less than 0.125 pg/ml, the subject does not have colorectal cancer.

For the method based on four proteins, it was determined that the following diagnostic method was performed:

i) if (6.7×[TRIM28])−(0.65×[PLOD1])−(13.13×[CEACAM5])+(0.43×[P4HA1]) −2.21 is less than zero, the subject has colorectal cancer; and

ii) if (6.7×[TRIM28])−(0.65×[PLOD1])−(13.13×[CEACAM5])+(0.43×[P4HA1]) −2.21 is greater than or equal to zero, the subject does not have colorectal cancer. The protein concentrations are utilised in this method in the following units: TRIM28, PLOD1 and

P4HA1 ng/ml, CEACAM5 in pg/ml.

1. A method of diagnosing colorectal cancer in a subject, comprising determining the concentrations of the proteins TRIM28, PLOD1 and CEACAM5 in a blood-derived sample from the subject, and based on the summed concentrations and/or relative concentrations of said proteins determining whether the subject is suffering from colorectal cancer.

2. The method of claim 1, further comprising determining the concentration of the protein P4HA1 in said sample, wherein the P4HA1 concentration is included in the determination of whether the subject is suffering from colorectal cancer.

3. The method of claim 1, wherein:
i) if the concentration of TRIM28 is greater than 0.3 ng/ml, the subject does not have colorectal cancer;
ii) if the concentration of TRIM28 is less than or equal to 0.3 ng/ml and the concentration of PLOD1 is greater than or equal to 1.7 ng/ml, the subject has colorectal cancer;
iii) if the concentration of TRIM28 is less than or equal to 0.3 ng/ml, the concentration of PLOD1 is less than 1.7 ng/ml and the concentration of CEACAM5 is greater than or equal to 0.13 pg/ml, the subject has colorectal cancer; and
iv) if the concentration of TRIM28 is less than or equal to 0.3 ng/ml, the concentration of PLOD1 is less than 1.7 ng/ml and the concentration of CEACAM5 is less than 0.13 pg/ml, the subject does not have colorectal cancer.

4. The method of claim 2, wherein:
i) if (6.7×[TRIM28])−(0.7×[PLOD1])−(13.1×[CEACAM5])+(0.4×[P4HA1])−2.2 is less than zero, the subject has colorectal cancer; and
ii) if (6.7×[TRIM28])−(0.7×[PLOD1])−(13.1×[CEACAM5])+(0.4×[P4HA1])−2.2 is greater than or equal to zero, the subject does not have colorectal cancer;
wherein the concentrations of TRIM28, PLOD1 and P4HA1 are measured in ng/ml, and the concentration of CEACAM5 is measured in pg/ml.

5. The method of any one of claims 1 to 4, further comprising taking a blood sample from the subject.

6. The method of any one of claims 1 to 5, wherein the blood-derived sample is a plasma sample.

7. The method of any one of claims 1 to 6, wherein the protein concentrations are measured by an immunoassay.

8. The method of claim 7, wherein said immunoassay is quantitative ELISA.

9. The method of any one of claims 1 to 8, wherein the steps of measuring the protein concentrations and determining whether the subject is suffering from colorectal cancer are performed by a computer processor programmed to perform said steps.

10. A method of diagnosing and treating colorectal cancer in a subject, comprising:
a) measuring the concentrations of the proteins TRIM28, PLOD1 and CEACAM5 in a blood-derived sample;
b) based on said concentrations, determining whether the subject is suffering from colorectal cancer; wherein:
i) if the concentration of TRIM28 is greater than 0.3 ng/ml, the subject does not have colorectal cancer;
ii) if the concentration of TRIM28 is less than or equal to 0.3 ng/ml and the concentration of PLOD1 is greater than or equal to 1.7 ng/ml, the subject has colorectal cancer;
iii) if the concentration of TRIM28 is less than or equal to 0.3 ng/ml, the concentration of PLOD1 is less than 1.7 ng/ml and the concentration of CEACAM5 is greater than or equal to 0.13 pg/ml, the subject has colorectal cancer; and
iv) if the concentration of TRIM28 is less than or equal to 0.3 ng/ml, the concentration of PLOD1 is less than 1.7 ng/ml and the concentration of CEACAM5 is less than 0.13 pg/ml, the subject does not have colorectal cancer; and
c) if the subject is diagnosed with colorectal cancer, administering treatment for colorectal cancer to the subject.

11. A method of diagnosing and treating colorectal cancer in a subject, comprising:
a) measuring the concentrations of the proteins TRIM28, PLOD1, CEACAM5 and P4HA1 in a blood-derived sample, wherein the concentrations of TRIM28, PLOD1 and P4HA1 are measured in ng/ml, and the concentration of CEACAM5 is measured in pg/ml; and
b) based on said concentrations, determining whether the subject is suffering from colorectal cancer; wherein:
i) if (6.7×[TRIM28])−(0.7×[PLOD1])−(13.1×[CEACAM5])+(0.4×[P4HA1]) −2.2 is less than zero, the subject has colorectal cancer; and
ii) if (6.7×[TRIM28])−(0.7×[PLOD1])−(13.1×[CEACAM5])+(0.4×[P4HA1]) −2.2 is greater than or equal to zero, the subject does not have colorectal cancer; and
c) if the subject is diagnosed with colorectal cancer, administering treatment for colorectal cancer to the subject.

12. The method of claim 10 or 11, wherein said treatment comprises surgery, chemotherapy, radiotherapy and/or immunotherapy.

13. A kit comprising a set of reagents for determining the presence or concentration of TRIM28, PLOD1 and CEACAM5 in a sample.

14. The kit of claim 13, further comprising a reagent for determining the presence or concentration of P4HA1 in a sample.

15. The kit of claim 13, said kit comprising:
i) a specific binding agent which binds TRIM28, or a fragment thereof;
ii) a specific binding agent which binds PLOD1, or a fragment thereof; and
iii) a specific binding agent which binds CEACAM5, or a fragment thereof.

16. The kit of claim 15, said kit further comprising a specific binding agent which binds P4HA1, or a fragment thereof.

17. The kit of claim 15 or 16, wherein each specific binding agent is an antibody.

18. The kit of any one of claims 13 to 17, wherein said kit is for performing an ELISA.

19. A computer programme product comprising instructions that, when executed, will cause a processor to perform a method as defined in any one of claims 1 to 4.

20. Use of a kit as defined in any one of claims 13 to 18 in the diagnosis of colorectal cancer, wherein said diagnosis is performed using a method as defined in any one of claims 1 to 9.


SEQUENCE LISTING (4 sequences)

SEQ ID NO: 1 - protein, 835 aa, Homo sapiens
MAASAAAASAAAASAASGSPGPGEGSAGGEKRSTAPSAAASASASAAASSPAGGGAEALELLEHCGVCRERLRPEREPRLLPCLHSACSACLGPAAPAAANSSGDGGAAGDGTVVDCPVCKQQCFSKDIVENYFMRDSGSKAATDAQDANQCCTSCEDNAPATSYCVECSEPLCETCVEAHQRVKYTKDHTVRSTGPAKSRDGERTVYCNVHKHEPLVLFCESCDTLTCRDCQLNAHKDHQYQFLEDAVRNQRKLLASLVKRLGDKHATLQKSTKEVRSSIRQVSDVQKRVQVDVKMAILQIMKELNKRGRVLVNDAQKVTEGQQERLERQHWTMTKIQKHQEHILRFASWALESDNNTALLLSKKLIYFQLHRALKMIVDPVEPHGEMKFQWDLNAWTKSAEAFGKIVAERPGTNSTGPAPMAPPRAPGPLSKQGSGSSQPMEVQEGYGFGSGDDPYSSAEPHVSGVKRSRSGEGEVSGLMRKVPRVSLERLDLDLTADSQPPVFKVFPGSTTEDYNLIVIERGAAAAATGQPGTAPAGTPGAPPLAGMAIVKEEETEAAIGAPPTATEGPETKPVLMALAEGPGAEGPRLASPSGSTSSGLEVVAPEGTSAPGGGPGTLDDSATICRVCQKPGDLVMCNQCEFCFHLDCHLPALQDVPGEEWSCSLCHVLPDLKEEDGSLSLDGADSTGVVAKLSPANQRKCERVLLALFCHEPCRPLHQLATDSTFSLDQPGGTLDLTLIRARLQEKLSPPYSSPQEFAQDVGRMFKQFNKLTEDKADVQSIIGLQRFFETRMNEAFGDTKFSAVLVEPPPMSLPGAGLSSQELSGGPGDGP

SEQ ID NO: 2 - protein, 727 aa, Homo sapiens
MRPLLLLALLGWLLLAEAKGDAKPEDNLLVLTVATKETEGFRRFKRSAQFFNYKIQALGLGEDWNVEKGTSAGGGQKVRLLKKALEKHADKEDLVILFADSYDVLFASGPRELLKKFRQARSQVVFSAEELIYPDRRLETKYPVVSDGKRFLGSGGFIGYAPNLSKLVAEWEGQDSDSDQLFYTKIFLDPEKREQINITLDHRCRIFQNLDGALDEVVLKFEMGHVRARNLAYDTLPVLIHGNGPTKLQLNYLGNYIPRFWTFETGCTVCDEGLRSLKGIGDEALPTVLVGVFIEQPTPFVSLFFQRLLRLHYPQKHMRLFIHNHEQHHKAQVEEFLAQHGSEYQSVKLVGPEVRMANADARNMGADLCRQDRSCTYYFSVDADVALTEPNSLRLLIQQNKNVIAPLMTRHGRLWSNFWGALSADGYYARSEDYVDIVQGRRVGVWNVPYISNIYLIKGSALRGELQSSDLFHHSKLDPDMAFCANIRQQDVFMFLTNRHTLGHLLSLDSYRTTHLHNDLWEVFSNPEDWKEKYIHQNYTKALAGKLVETPCPDVYWFPIFTEVACDELVEEMEHFGQWSLGNNKDNRIQGGYENVPTIDIHMNQIGFEREWHKFLLEYIAPMTEKLYPGYYTRAQFDLAFVVRYKPDEQPSLMPHHDASTFTINIALNRVGVDYEGGGCRFLRYNCSIRAPRKGWTLMHPGRLTHYHEGLPTTRGTRYIAVSFVDP

SEQ ID NO: 3 - protein, 702 aa, Homo sapiens
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNKLSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSAGATVGIMIGVLVGVALI

SEQ ID NO: 4 - protein, 534 aa, Homo sapiens
MIWYILIIGILLPQSLAHPGFFTSIGQMTDLIHTEKDLVTSLKDYIKAEEDKLEQIKKWAEKLDRLTSTATKDPEGFVGHPVNAFKLMKRLNTEWSELENLVLKDMSDGFISNLTIQRQYFPNDEDQVGAAKALLRLQDTYNLDTDTISKGNLPGVKHKSFLTAEDCFELGKVAYTEADYYHTELWMEQALRQLDEGEISTIDKVSVLDYLSYAVYQQGDLDKALLLTKKLLELDPEHQRANGNLKYFEYIMAKEKDVNKSASDDQSDQKTTPKKKGVAVDYLPERQKYEMLCRGEGIKMTPRRQKKLFCRYHDGNRNPKFILAPAKQEDEWDKPRIIRFHDIISDAEIEIVKDLAKPRLRRATISNPITGDLETVHYRISKSAWLSGYENPVVSRINMRIQDLTGLDVSTAEELQVANYGVGGQYEPHFDFARKDEPDAFKELGTGNRIATWLFYMSDVSAGGATVFPEVGASVWPKKGTAVFWYNLFASGEGDYSTRHAACPVLVGNKWVSNKWLHERGQEFRRPCTLSELE